(19) United States
(12) Patent Application Publication
(10) Pub. No.: US 2025/0087351 A1
(43) Pub. Date: Mar. 13, 2025

(54) ORDERED CODE SEQUENCES USING A COMPOSITE MACHINE LEARNING MODEL

(71) Applicant: Optum, Inc., Minnetonka, MN (US)

(72) Inventors: YiZi Xiao, Lakeville, MN (US); Mohammed Mahmood Modan, Silverthorne, CO (US); Gopathy Purushothaman, Nashville, TN (US); Tamara Balac Sipes, San Diego, CA (US); Vijay S Nori, Roswell, GA (US); Ryan Michael Allen, Onalaska, WI (US)

(21) Appl. No.: 18/462,961

(22) Filed: Sep. 7, 2023

(51) Int. Cl. G16H 50/20; G06N 3/09

(52) U.S. Cl. CPC G16H 50/20; G06N 3/09

(57) ABSTRACT

Various embodiments of the present disclosure provide techniques for generating an ordered code sequence. For example, the techniques may include generating a predictive group code and an anchor code for an entity based on entity data. The techniques may include generating, using the first portion of the composite machine learning model, an unordered code sequence comprising one or more predicted codes based on the entity data, the predictive group code, and the anchor code. The techniques may include generating, using a second portion of the composite machine learning model, an ordered code sequence based on the unordered code sequence. The techniques may include providing the ordered code sequence.

### BACKGROUND

Various embodiments of the present disclosure address technical challenges related to generating ordered code sequences given limitations of existing machine learning and/or rules-based model approaches. Existing techniques for generating ordered code sequences are unable to leverage multiple different machine learning and/or rules-based models to generate an ordered code sequence from input data in an efficient, accurate, and scalable manner. Various embodiments of the present disclosure make important contributions to various existing techniques for generating ordered code sequences by addressing these technical challenges.

### BRIEF SUMMARY

Various embodiments of the present disclosure disclose a composite machine learning model and methods for generating an ordered code sequence. Conventional techniques for generating an ordered code sequence implement an individual conventional machine learning model and/or rules-based model to generate ordered code sequences based on input data. However, these conventional techniques are inefficient (e.g., resource intensive and/or time consuming), lack scalability for large ordered sequences and/or large quantities of input data, and/or generate inaccurate ordered code sequences. The present disclosure provides a composite machine learning model that leverages multiple different machine learning and/or rules-based models that are jointly trained to generate an ordered code sequence. In this way, using the techniques of the present disclosure, a composite machine learning model may be leveraged to generate an ordered code sequence in a more efficient, accurate, and scalable manner than conventional techniques for generating an ordered code sequence using structured and/or unstructured input data.

In some embodiments, a computer-implemented method comprises generating, by one or more processors and using a first portion of a composite machine learning model, a predictive group code and an anchor code for an entity based on entity data; generating, by the one or more processors and using the first portion of the composite machine learning model, an unordered code sequence comprising one or more predicted codes based on the entity data, the predictive group code, and the anchor code; generating, by the one or more processors and using a second portion of the composite machine learning model, an ordered code sequence based on the unordered code sequence; and providing, by the one or more processors, the ordered code sequence.

In some embodiments, a computing system comprises a memory and one or more processors communicatively coupled to the memory. The one or more processors are configured to generate, using a first portion of a composite machine learning model, a predictive group code and an anchor code for an entity based on entity data; generate, using the first portion of the composite machine learning model, an unordered code sequence comprising one or more predicted codes based on the entity data, the predictive group code, and the anchor code; generate, using a second portion of the composite machine learning model, an ordered code sequence based on the unordered code sequence; and provide the ordered code sequence.

In some embodiments, one or more non-transitory computer-readable storage media include instructions that, when executed by one or more processors, cause the one or more processors to generate, using a first portion of a composite machine learning model, a predictive group code and an anchor code for an entity based on entity data; generate, using the first portion of the composite machine learning model, an unordered code sequence comprising one or more predicted codes based on the entity data, the predictive group code, and the anchor code; generate, using a second portion of the composite machine learning model, an ordered code sequence based on the unordered code sequence; and provide the ordered code sequence.

### BRIEF DESCRIPTION OF THE DRAWINGS

FIG. 1 illustrates an example computing system in accordance with one or more embodiments of the present disclosure;

FIG. 2 is a schematic diagram showing a system computing architecture in accordance with some embodiments discussed herein;

FIG. 3 is a block diagram of an example system architecture of a composite machine learning model in accordance with some embodiments discussed herein;

FIG. 4 is a dataflow diagram showing example data structures for generating an ordered code sequence in accordance with some embodiments discussed herein; and

FIG. 5 is a flowchart showing an example of a process for generating ordered code sequence in accordance with some embodiments discussed herein.

### DETAILED DESCRIPTION

Various embodiments of the present disclosure are described more fully hereinafter with reference to the accompanying drawings, in which some, but not all embodiments of the present disclosure are shown. Indeed, the present disclosure may be embodied in many different forms and should not be construed as limited to the embodiments set forth herein; rather, these embodiments are provided so that the present disclosure will satisfy applicable legal requirements. The term “or” is used herein in both the alternative and conjunctive sense, unless otherwise indicated. The terms “illustrative” and “example” are used to be examples with no indication of quality level. Terms such as “computing,” “determining,” “generating,” and/or similar words are used herein interchangeably to refer to the creation, modification, or identification of data. Further, “based on,” “based at least in part on,” “based at least on,” “based upon,” and/or similar words are used herein interchangeably in an open-ended manner such that they do not necessarily indicate being based only on or based solely on the referenced element or elements unless so indicated. Like numbers refer to like elements throughout.

### I. Computer Program Products, Methods, and Computing Entities

Embodiments of the present disclosure may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together, such as in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In some embodiments, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In some embodiments, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present disclosure may also be implemented as methods, apparatus, systems, computing devices, computing entities, and/or the like. As such, embodiments of the present disclosure may take the form of an apparatus, system, computing device, computing entity, and/or the like executing instructions stored on a computer-readable storage medium to perform certain steps or operations. Thus, embodiments of the present disclosure may also take the form of an entirely hardware embodiment, an entirely computer program product embodiment, and/or an embodiment that comprises combination of computer program products and hardware performing certain steps or operations.

Embodiments of the present disclosure are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some example embodiments, retrieval, loading, and/or execution may be performed in parallel such that multiple instructions are retrieved, loaded, and/or executed together. Thus, such embodiments may produce specifically-configured machines performing the steps or operations specified in the block diagrams and flowchart illustrations. Accordingly, the block diagrams and flowchart illustrations support various combinations of embodiments for performing the specified instructions, operations, or steps.

### II. Example Framework

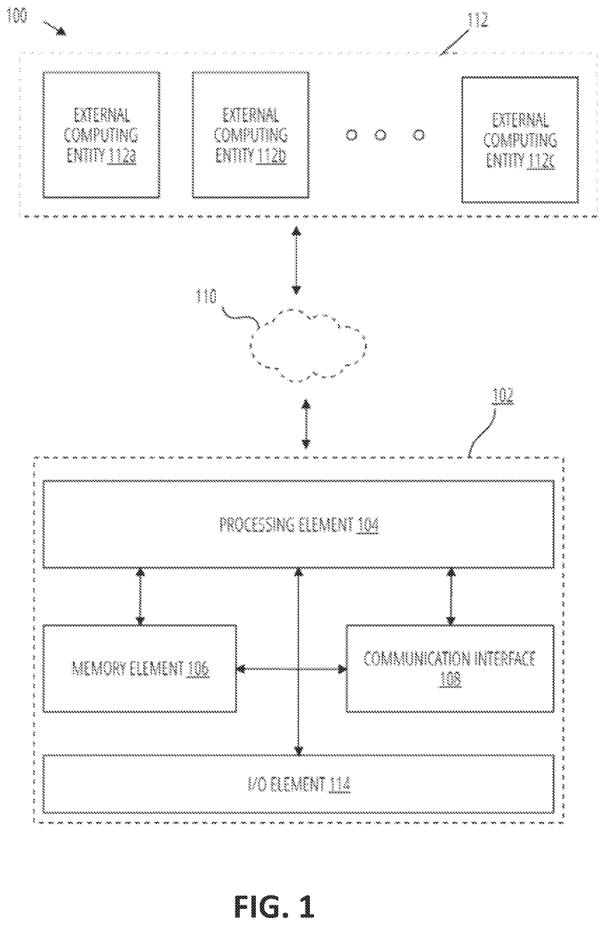

FIG. 1 illustrates an example computing system 100 in accordance with one or more embodiments of the present disclosure. The computing system 100 may include a predictive computing entity 102 and/or one or more external computing entities 112a-c communicatively coupled to the predictive computing entity 102 using one or more wired and/or wireless communication techniques. The predictive computing entity 102 may be specially configured to perform one or more steps/operations of one or more techniques described herein. In some embodiments, the predictive computing entity 102 may include and/or be in association with one or more mobile device(s), desktop computer(s), laptop(s), server(s), cloud computing platform(s), and/or the like. In some example embodiments, the predictive computing entity 102 may be configured to receive and/or transmit one or more datasets, objects, and/or the like from and/or to the external computing entities 112a-c to perform one or more steps/operations of one or more techniques (e.g., training techniques, data generation techniques, and/or the like) described herein.

The external computing entities 112a-c, for example, may include and/or be associated with one or more data centers, computing entities, and/or any other external entity that may be configured to receive, store, and/or interpret ordered code sequences. The data centers, for example, may be associated with one or more data repositories storing entity data, unordered code sequences, ordered code sequences and/or the like that can, in some circumstances, be processed by the predictive computing entity 102 to generate an ordered code sequence. The one or more of the external computing entities 112a-c may include one or more data processing entities that may receive, store, and/or have access to training data for machine learning models. The data processing entities, for example, may maintain a training datastore with a plurality of training ordered code sequences, training entity data, and/or the like.

The predictive computing entity 102 may include, or be in communication with, one or more processing elements 104 (also referred to as processors, processing circuitry, digital circuitry, and/or similar terms used herein interchangeably) that communicate with other elements within the predictive computing entity 102 via a bus, for example. As will be understood, the predictive computing entity 102 may be embodied in a number of different ways. The predictive computing entity 102 may be configured for a particular use or configured to execute instructions stored in volatile or non-volatile media or otherwise accessible to the processing element 104. As such, whether configured by hardware or computer program products, or by a combination thereof, the processing element 104 may be capable of performing steps or operations according to embodiments of the present disclosure when configured accordingly.

In one embodiment, the predictive computing entity 102 may further include, or be in communication with, one or more memory elements 106. The memory element 106 may be used to store at least portions of the databases, database instances, database management systems, data, applications, programs, program modules, scripts, source code, object code, byte code, compiled code, interpreted code, machine code, executable instructions, and/or the like being executed by, for example, the processing element 104. Thus, the databases, database instances, database management systems, data, applications, programs, program modules, scripts, source code, object code, byte code, compiled code, interpreted code, machine code, executable instructions, and/or the like may be used to control certain aspects of the operation of the predictive computing entity 102 with the assistance of the processing element 104.

As indicated, in one embodiment, the predictive computing entity 102 may also include one or more communication interfaces 108 for communicating with various computing entities, e.g., external computing entities 112a-c, such as by communicating data, content, information, and/or similar terms used herein interchangeably that may be transmitted, received, operated on, processed, displayed, stored, and/or the like.

The computing system 100 may include one or more input/output (I/O) element(s) 114 for communicating with one or more users. An I/O element 114, for example, may include one or more user interfaces for providing and/or receiving information from one or more users of the computing system 100. The I/O element 114 may include one or more tactile interfaces (e.g., keypads, touch screens, etc.), one or more audio interfaces (e.g., microphones, speakers, etc.), visual interfaces (e.g., display devices, etc.), and/or the like. The I/O element 114 may be configured to receive user input through one or more of the user interfaces from a user of the computing system 100 and provide data to a user through the user interfaces.

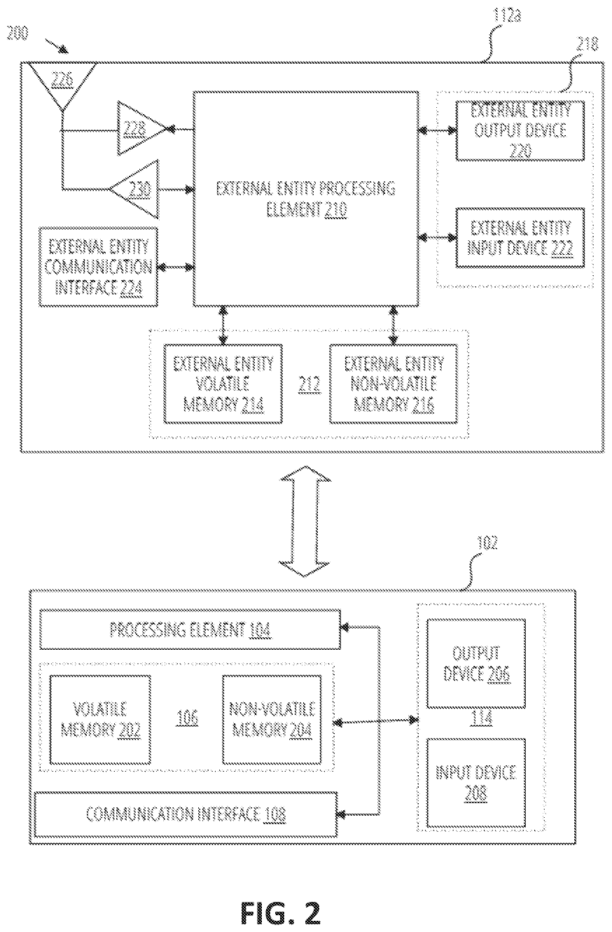

FIG. 2 is a schematic diagram showing a system computing architecture 200 in accordance with some embodiments discussed herein. In some embodiments, the system computing architecture 200 may include the predictive computing entity 102 and/or the external computing entity 112a of the computing system 100. The predictive computing entity 102 and/or the external computing entity 112a may include a computing system, a computing apparatus, a computing device, and/or any form of computing entity configured to execute instructions stored on a computer-readable storage medium to perform certain steps or operations.

The predictive computing entity 102 may include a processing element 104, a memory element 106, a communication interface 108, and/or one or more I/O elements 114 that communicate within the predictive computing entity 102 via internal communication circuitry, such as a communication bus and/or the like.

The processing element 104 may be embodied as one or more complex programmable logic devices (CPLDs), microprocessors, multi-core processors, coprocessing entities, application-specific instruction-set processors (ASIPs), microcontrollers, and/or controllers. Further, the processing element 104 may be embodied as one or more other processing devices or circuitry including, for example, a processor, one or more processors, various processing devices and/or the like. The term circuitry may refer to an entirely hardware embodiment or a combination of hardware and computer program products. Thus, the processing element 104 may be embodied as integrated circuits, application specific integrated circuits (ASICs), field programmable gate arrays (FPGAs), programmable logic arrays (PLAs), hardware accelerators, digital circuitry, and/or the like.

The memory element 106 may include volatile memory 202 and/or non-volatile memory 204. The memory element 106, for example, may include volatile memory 202 (also referred to as volatile storage media, memory storage, memory circuitry and/or similar terms used herein interchangeably). In one embodiment, a volatile memory 202 may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

The memory element 106 may include non-volatile memory 204 (also referred to as non-volatile storage, memory, memory storage, memory circuitry and/or similar terms used herein interchangeably). In one embodiment, the non-volatile memory 204 may include one or more non-volatile storage or memory media, including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

In one embodiment, a non-volatile memory 204 may include a floppy disk, flexible disk, hard disk, solid-state storage (SSS) (e.g., a solid-state drive (SSD)), solid state card (SSC), solid state module (SSM), enterprise flash drive, magnetic tape, or any other non-transitory magnetic medium, and/or the like. A non-volatile memory 204 may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile memory 204 may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

As will be recognized, the non-volatile memory 204 may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity-relationship model, object model, document model, semantic model, graph model, and/or the like.

The memory element 106 may include a non-transitory computer-readable storage medium for implementing one or more aspects of the present disclosure including as a computer-implemented method configured to perform one or more steps/operations described herein. For example, the non-transitory computer-readable storage medium may include instructions that when executed by a computer (e.g., processing element 104), cause the computer to perform one or more steps/operations of the present disclosure. For instance, the memory element 106 may store instructions that, when executed by the processing element 104, configure the predictive computing entity 102 to perform one or more step/operations described herein.

Embodiments of the present disclosure may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language, such as an assembly language associated with a particular hardware framework and/or operating system platform. A software component comprising assembly language instructions may require conversion into executable machine code by an assembler prior to execution by the hardware framework and/or platform. Another example programming language may be a higher-level programming language that may be portable across multiple frameworks. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query, or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together, such as in a particular directory, folder, or library. Software components may be static (e.g., pre-established or fixed) or dynamic (e.g., created or modified at the time of execution).

The predictive computing entity 102 may be embodied by a computer program product include non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media such as the volatile memory 202 and/or the non-volatile memory 204.

The predictive computing entity 102 may include one or more I/O elements 114. The I/O elements 114 may include one or more output devices 206 and/or one or more input devices 208 for providing and/or receiving information with a user, respectively. The output devices 206 may include one or more sensory output devices, such as one or more tactile output devices (e.g., vibration devices such as direct current motors, and/or the like), one or more visual output devices (e.g., liquid crystal displays, and/or the like), one or more audio output devices (e.g., speakers, and/or the like), and/or the like. The input devices 208 may include one or more sensory input devices, such as one or more tactile input devices (e.g., touch sensitive displays, push buttons, and/or the like), one or more audio input devices (e.g., microphones, and/or the like), and/or the like.

In addition, or alternatively, the predictive computing entity 102 may communicate, via a communication interface 108, with one or more external computing entities such as the external computing entity 112a. The communication interface 108 may be compatible with one or more wired and/or wireless communication protocols.

For example, such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. In addition, or alternatively, the predictive computing entity 102 may be configured to communicate via wireless external communication using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.9 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

The external computing entity 112a may include an external entity processing element 210, an external entity memory element 212, an external entity communication interface 224, and/or one or more external entity I/O elements 218 that communicate within the external computing entity 112a via internal communication circuitry, such as a communication bus and/or the like.

The external entity processing element 210 may include one or more processing devices, processors, and/or any other device, circuitry, and/or the like described with reference to the processing element 104. The external entity memory element 212 may include one or more memory devices, media, and/or the like described with reference to the memory element 106. The external entity memory element 212, for example, may include at least one external entity volatile memory 214 and/or external entity non-volatile memory 216. The external entity communication interface 224 may include one or more wired and/or wireless communication interfaces as described with reference to communication interface 108.

In some embodiments, the external entity communication interface 224 may be supported by one or more radio circuitry. For instance, the external computing entity 112a may include an antenna 226, a transmitter 228 (e.g., radio), and/or a receiver 230 (e.g., radio).

Signals provided to and received from the transmitter 228 and the receiver 230, correspondingly, may include signaling information/data in accordance with air interface standards of applicable wireless systems. In this regard, the external computing entity 112a may be capable of operating with one or more air interface standards, communication protocols, modulation types, and access types. More particularly, the external computing entity 112a may operate in accordance with any of a number of wireless communication standards and protocols, such as those described above with regard to the predictive computing entity 102.

Via these communication standards and protocols, the external computing entity 112a may communicate with various other entities using means such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The external computing entity 112a may also download changes, add-ons, and updates, for instance, to its firmware, software (e.g., including executable instructions, applications, program modules), operating system, and/or the like.

According to one embodiment, the external computing entity 112a may include location determining embodiments, devices, modules, functionalities, and/or the like. For example, the external computing entity 112a may include outdoor positioning embodiments, such as a location module adapted to acquire, for example, latitude, longitude, altitude, geocode, course, direction, heading, speed, universal time (UTC), date, and/or various other information/data. In one embodiment, the location module may acquire data, such as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data may be collected using a variety of coordinate systems, such as the DecimalDegrees (DD); Degrees, Minutes, Seconds (DMS); Universal Transverse Mercator (UTM); Universal Polar Stereographic (UPS) coordinate systems; and/or the like. Alternatively, the location information/data may be determined by triangulating a position of the external computing entity 112a in connection with a variety of other systems, including cellular towers, Wi-Fi access points, and/or the like. Similarly, the external computing entity 112a may include indoor positioning embodiments, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning embodiments may be used in a variety of settings to determine the location of someone or something to within inches or centimeters.

The external entity I/O elements 218 may include one or more external entity output devices 220 and/or one or more external entity input devices 222 that may include one or more sensory devices described herein with reference to the I/O elements 114. In some embodiments, the external entity I/O element 218 may include a user interface (e.g., a display, speaker, and/or the like) and/or a user input interface (e.g., keypad, touch screen, microphone, and/or the like) that may be coupled to the external entity processing element 210.

For example, the user interface may be a user application, browser, and/or similar words used herein interchangeably executing on and/or accessible via the external computing entity 112a to interact with and/or cause the display, announcement, and/or the like of information/data to a user. The user input interface may include any of a number of input devices or interfaces allowing the external computing entity 112a to receive data including, as examples, a keypad (hard or soft), a touch display, voice/speech interfaces, motion interfaces, and/or any other input device. In embodiments including a keypad, the keypad may include (or cause display of) the conventional numeric (0-9) and related keys (#, *, and/or the like), and other keys used for operating the external computing entity 112a and may include a full set of alphabetic keys or set of keys that may be activated to provide a full set of alphanumeric keys. In addition to providing input, the user input interface may be used, for example, to activate or deactivate certain functions, such as screen savers, sleep modes, and/or the like.

### III. Examples of Certain Terms

In some embodiments, the term “entity data” refers to a data entity that describes data provided to an algorithm to generate a predictive output. The type, format, and parameters of the data entity may be based on the prediction domain. Entity data may include a plurality of entity data values that may be considered by an algorithm to generate the predictive output. In some embodiments, the algorithm is a machine learning model that may be trained to generate the predictive output based on the entity data values of the entity data. As one example, in an ordered code sequence prediction domain, entity data may correspond to one or more medical records associated with a patient. In such a case, entity data may include one or more entity data values that describe a patient's prior procedures, prior diagnosis related group codes, prior current procedural terminology codes, prior international statistical classification of diseases codes, demographic features, and/or the like, and/or any other value descriptive of an ordered code sequence process.

In some embodiments, the term “entity” refers to a data entity that describes an entity corresponding to entity data. The type, format, and parameters of the entity may be based on the prediction domain. An entity may include a plurality of entity values that may be considered by an algorithm to generate a predictive output. In some examples, the algorithm may include a machine learning model that is trained to generate a predictive output for entity data based on one or more entity values of a corresponding entity. As one example, in an ordered code sequence prediction domain, an entity may correspond to a patient for which an ordered code sequence may be generated.

In some embodiments, the term “machine learning predictive group code model” refers to a data entity that describes parameters, hyper-parameters, and/or defined operations of a rules-based and/or machine learning model (e.g., model including at least one of one or more rule-based layers, one or more layers that depend on trained parameters, coefficients, and/or the like). The machine learning predictive group code model may be configured to generate a predictive group code based on entity data. The machine learning predictive group code model may include one or more of any type of machine learning model including one or more supervised, unsupervised, semi-supervised, and/or reinforcement learning models. As an example, the machine learning predictive group code model may include a gradient boosted random forest machine learning model configured to generate a predictive group code based on entity data. The machine learning predictive group code model may include multiple models configured to generate a predictive group code based on entity data.

In some embodiments, the term “predictive group code” refers to code generated using a machine learning predictive group code model. As one example, in an ordered code sequence prediction domain, a predictive group code may correspond to a diagnosis related group code corresponding to an entity for which an ordered code sequence may be generated. For example, a predictive group code may correspond to an outpatient procedure associated with an entity for which an ordered code sequence may be generated.

In some embodiments, the term “machine learning anchor code model” refers to a data entity that describes parameters, hyper-parameters, and/or defined operations of a rules-based and/or machine learning model (e.g., model including at least one of one or more rule-based layers, one or more layers that depend on trained parameters, coefficients, and/or the like). The machine learning anchor code model may be configured to generate an anchor code based on entity data. The machine learning anchor code model may include one or more of any type of machine learning model including one or more supervised, unsupervised, semi-supervised, and/or reinforcement learning models. As an example, the machine learning anchor code model may include a gradient boosted random forest machine learning model configured to generate an anchor code based on entity data. The machine learning anchor code model may include multiple models configured to generate an anchor code based on entity data.

In some embodiments, the term “anchor code” refers to code generated using a machine learning anchor code model. In some embodiments, an anchor code is the first code in an ordered sequence. As one example, in an ordered code sequence prediction domain, an anchor code may correspond to a current procedural terminology code and/or an international statistical classification of diseases code corresponding to an entity for which an ordered code sequence may be generated.

In some embodiments, the term “machine learning code sequence model” refers to a data entity that describes parameters, hyper-parameters, and/or defined operations of a rules-based and/or machine learning model (e.g., model including at least one of one or more rule-based layers, one or more layers that depend on trained parameters, coefficients, and/or the like). A machine learning code sequence model may be configured to generate an unordered code sequence based on entity data, an anchor code, and a predictive group code. The machine learning code sequence model may include one or more of any type of machine learning model including one or more supervised, unsupervised, semi-supervised, and/or reinforcement learning models. In some embodiments, the machine learning code sequence model includes multiple models configured to perform one or more different stages of generating an unordered code sequence based on entity data, an anchor code, and a predictive group code. The machine learning code sequence model may include an embedding layer, a recurrent neural network layer, a linear layer, and a prediction layer.

In some embodiments, the term “embedding layer” refers to a data entity that describes parameters, hyper-parameters, and/or defined operations of a rules-based and/or machine learning model (e.g., model including at least one of one or more rule-based layers, one or more layers that depend on trained parameters, coefficients, and/or the like). The embedding layer may be one of a plurality of layers of the machine learning code sequence model. The embedding layer may be configured to generate refined entity data based on at least a portion of the entity data. For example, entity data may correspond to a sparse vector Vᵢₙ^{enc}ϵR^{n×1} where n represents a number of entity data values in the entity data and the refined entity data may correspond to a dense vector V_{emb}^{enc}ϵR^{m×1} where n>>m and m represents a portion of the number of entity data values in the refined entity data (e.g., the embedding layer may be configured to map a sparse vector to a dense vector). As an example, in an ordered code sequence prediction domain, n may represent a total number of a patient's prior procedures, prior diagnosis related group codes, prior current procedural terminology codes, prior international statistical classification of diseases codes, demographic features, and/or the like, and/or any other value descriptive of an ordered code sequence process. As an example, in an ordered code sequence prediction domain, m may represent a portion of the number of a patient's prior procedures, prior diagnosis related group codes, prior current procedural terminology codes, prior international statistical classification of diseases codes, demographic features, and/or the like, and/or any other value.

In some embodiments, the term “refined entity data” refers to a data entity that describes data provided to an algorithm to generate a predictive output. The type, format, and parameters of the data entity may be based on the prediction domain. Refined entity data may include a plurality of refined entity data values that may be considered by an algorithm to generate the predictive output. In some embodiments, the algorithm is a machine learning model that may be trained to generate the predictive output based on the refined entity data values of the refined entity data. As one example, in an ordered code sequence prediction domain, refined entity data may correspond to a portion of one or more medical records associated with a patient. In such a case, refined entity data may include one or more refined entity data values that describe a portion of a patient's prior procedures, prior diagnosis related group codes, prior current procedural terminology codes, prior international statistical classification of diseases codes, demographic features, and/or the like, and/or any other value descriptive of an ordered code sequence process.

In some embodiments, the term “recurrent neural network layer” refers to a data entity that describes parameters, hyper-parameters, and/or defined operations of a rules-based and/or machine learning model (e.g., model including at least one of one or more rule-based layers, one or more layers that depend on trained parameters, coefficients, and/or the like). The recurrent neural network layer may be one of a plurality of layers of the machine learning code sequence model. The recurrent neural network layer may be configured to identify a plurality of predicted codes based on the refined entity data. The recurrent neural network may include a plurality of layers. The recurrent neural network layer may generate a hidden state vector V_{hid}^{enc} and an output vector Vₒᵤₜ^{enc} for each of the plurality of layers and the recurrent neural network layer may concatenate the hidden state vector V_{hid}^{enc} from the last layer of the plurality of layers with a R^{2×1} vector to generate a context vector. The context vector may represent the plurality of predicted codes. The R^{2×1} vector may represent at least a portion of the entity data. In some embodiments, the recurrent neural network layer is bidirectional. In some embodiments, the recurrent neural network layer comprises a gated recurrent unit.

In some embodiments, the term “plurality of predicted codes” refers to a collection of one or more codes. As one example, in an ordered code sequence prediction domain, a plurality of predicted codes may include diagnosis related group codes, current procedural terminology codes, and/or international statistical classification of diseases codes.

In some embodiments, the term “linear layer” refers to a data entity that describes parameters, hyper-parameters, and/or defined operations of a rules-based and/or machine learning model (e.g., model including at least one of one or more rule-based layers, one or more layers that depend on trained parameters, coefficients, and/or the like). The linear layer may be configured to identify a number of predicted codes in an unordered code sequence based on a plurality of predicted codes and at least a portion of the entity data. As one example, in an ordered code sequence prediction domain, the linear layer may identify a number of diagnosis related group codes, current procedural terminology codes, and/or international statistical classification of diseases codes in an unordered code sequence.

In some embodiments, the term “prediction layer” refers to a data entity that describes parameters, hyper-parameters, and/or defined operations of a rules-based and/or machine learning model (e.g., model including at least one of one or more rule-based layers, one or more layers that depend on trained parameters, coefficients, and/or the like). As an example, the prediction layer may include a classification model. As an example, the prediction layer may include a regression model.

In some embodiments, the term “classification model” refers to a data entity that describes parameters, hyper-parameters, and/or defined operations of a rules-based and/or machine learning model (e.g., model including at least one of one or more rule-based layers, one or more layers that depend on trained parameters, coefficients, and/or the like). The classification model may be configured to identify one or more predicted codes in an unordered code sequence from a plurality of predicted codes. For example, the classification model may be configured to identify one or more predicted codes in an unordered code sequence from a plurality of predicted codes using a sigmoid activation function. The classification model (e.g., using a sigmoid activation function) may be configured to identify the one or more predicted codes in an unordered code sequence from a plurality of codes by determining which of the plurality of codes is associated with a probability of being in an unordered code sequence that exceeds a probability threshold. For example, the classification model (e.g., using a sigmoid activation function) may be configured to identify the one or more predicted codes in an unordered code sequence from a plurality of codes by determining which of the plurality of codes is associated with a probability of being in an unordered code sequence that exceeds a probability threshold of 0.5. In some embodiments, the classification model is trained using a binary cross-entropy technique. Additionally, or alternatively, the classification model is trained using a root-mean-squared error technique. As one example, in an ordered code sequence prediction domain, the classification model may identify one or more diagnosis related group codes, current procedural terminology codes, and/or international statistical classification of diseases codes in an unordered code sequence.

In some embodiments, the term “regression model” refers to a data entity that describes parameters, hyper-parameters, and/or defined operations of a rules-based and/or machine learning model (e.g., model including at least one of one or more rule-based layers, one or more layers that depend on trained parameters, coefficients, and/or the like). The regression model may be configured to determine an occurrence of each of one or more predicted codes in an unordered code sequence. For example, the regression model may be configured to determine an occurrence of each of the one or more predicted codes in an unordered code sequence using a rectified linear unit activation function. The regression model (e.g., using a rectified linear unit activation function) may be configured to determine an occurrence of each of one or more predicted codes in an unordered code sequence by determining a count associated with each code in an unordered code sequence. For example, the regression model (e.g., using a rectified linear unit activation function) may be configured to determine an occurrence of a predicted code in an unordered code sequence by determining that the predicted code is associated with a count of 3 (e.g., the predicted code occurs 3 times in the unordered code sequence). In some embodiments, the regression model is trained using a binary cross-entropy technique. Additionally, or alternatively, the regression model is trained using a root-mean-squared error technique. As one example, in an ordered code sequence prediction domain, the regression model may determine an occurrence of each of one or more diagnosis related group codes, current procedural terminology codes, and/or international statistical classification of diseases codes in an unordered code sequence.

In some embodiments, the term “unordered code sequence” refers to a sequence of codes generated by the machine learning code sequence model that are not organized in a particular determined order. As one example, in an ordered code sequence prediction domain, an unordered code sequence may include a sequence of diagnosis related group codes, current procedural terminology codes, and/or international statistical classification of diseases codes that are not organized in a particular determined order.

In some embodiments, the term “graphical model” refers to a data entity that describes parameters, hyper-parameters, and/or defined operations of a rules-based and/or machine learning model (e.g., model including at least one of one or more rule-based layers, one or more layers that depend on trained parameters, coefficients, and/or the like). The graphical model may be configured to generate an ordered code sequence based on an unordered code sequence. The graphical model may include one or more of any type of machine learning model including one or more supervised, unsupervised, semi-supervised, and/or reinforcement learning models. As an example, the graphical model may include a transition probably matrix configured to generate the ordered code sequence. The graphical model may include multiple models configured to generate the ordered code sequence based on the unordered code sequence.

In some embodiments, the term “ordered code sequence” refers to a sequence of codes that are organized in a particular determined order. As one example, in an ordered code sequence prediction domain, an ordered code sequence may include a sequence of diagnosis related group codes, current procedural terminology codes, and/or international statistical classification of diseases codes that are organized in a particular determined order.

In some embodiments, the term “one or more predicted codes” refers to codes in an unordered code sequence and/or an ordered code sequence. As one example, in an ordered code sequence prediction domain, one or more predicted codes may include diagnosis related group codes, current procedural terminology codes, and/or international statistical classification of diseases codes in an unordered code sequence and/or an ordered code sequence. In some embodiments, the one or more predicted codes are distinct from the plurality of predicted codes in that all of the one or more predicted codes are in an unordered code sequence and/or an ordered code sequence while not all of the plurality of predicted codes are in an unordered code sequence and/or an ordered code sequence.

In some embodiments, the term “composite machine learning model” refers to a data entity that describes parameters, hyper-parameters, and/or defined operations of a rules-based and/or machine learning model (e.g., model including at least one of one or more rule-based layers, one or more layers that depend on trained parameters, coefficients, and/or the like). A composite machine learning model may be configured to generate an ordered code sequence based on entity data. The composite machine learning model may include one or more of any type of machine learning model including one or more supervised, unsupervised, semi-supervised, and/or reinforcement learning models. The composite machine learning model may include multiple models configured to perform one or more different stages of generating an ordered code sequence based on entity data. As an example, the composite machine learning model may include a machine learning predictive group code model, a machine learning anchor code model, a machine learning code sequence model, and/or a graphical model. As an example, the composite machine learning model may include a first portion and a second portion.

In some embodiments, the term “first portion” refers to a data entity that describes parameters, hyper-parameters, and/or defined operations of a portion of a rules-based and/or machine learning model (e.g., model including at least one of one or more rule-based layers, one or more layers that depend on trained parameters, coefficients, and/or the like). For example, the first portion may refer to a first portion of the composite machine learning model. The first portion of the composite machine learning model may be configured to generate an unordered code sequence based on entity data. The first portion of the composite machine learning model may include one or more of any type of machine learning model including one or more supervised, unsupervised, semi-supervised, and/or reinforcement learning models. The first portion of the composite machine learning model may include multiple models configured to perform one or more different stages of generating an unordered code sequence based on entity data. As an example, the first portion of the composite machine learning model may include a machine learning predictive group code model, a machine learning anchor code model, and/or a machine learning code sequence model.

In some embodiments, the term “second portion” refers to a data entity that describes parameters, hyper-parameters, and/or defined operations of a portion of a rules-based and/or machine learning model (e.g., model including at least one of one or more rule-based layers, one or more layers that depend on trained parameters, coefficients, and/or the like). For example, the second portion may refer to a second portion of the composite machine learning model The second portion of the composite machine learning model may be configured to generate an unordered code sequence based on entity data. The second portion of the composite machine learning model may include one or more of any type of machine learning model including one or more supervised, unsupervised, semi-supervised, and/or reinforcement learning models. The second portion of the composite machine learning model may include multiple models configured to perform one or more different stages of generating an ordered code sequence based on an unordered code sequence. As an example, the second portion of the composite machine learning model may include a graphical model.

### IV. Overview

Embodiments of the present disclosure present machine learning and data generation techniques that improve computer generation of ordered code sequences. To do so, the present disclosure provides a composite machine learning model that leverages multiple types of machine learning models and/or rules-based models to generate an ordered code sequence based on input data in an efficient, accurate, and scalable manner. In this way, the present disclosure provides a new composite machine learning model and methods for implementing techniques for generating ordered code sequences that improve upon conventional ordered code generation techniques.

The composite machine learning model may be applied to entity data as well as any other unstructured and/or structured input data to generate an ordered code sequence. To do so, the composite machine learning model includes a machine learning predictive group code model configured to generate a predictive group code based on input data, such as entity data. The predictive group code may be one of the codes in the ordered code sequence. The composite machine learning model includes a machine learning anchor code model configured to generate an anchor code based on input data. The anchor code may be the first code in the ordered code sequence. The composite machine learning model may include a machine learning code sequence model configured to generate an unordered code sequence based on the input data, the predictive group code, and the anchor code. The unordered code sequence may include the predictive group code, the anchor code, and one or more other codes. The machine learning code sequence model may include an embedding layer, a recurrent neural network layer, a linear layer, and a classification layer. The composite machine learning model may include a graphical model configured to generate an ordered code sequence based on the unordered code sequence. The ordered code sequence may include the codes in the unorganized code sequence organized into a particular order.

The present disclosure is novel compared to known solutions in the following ways: (1) uses historical claims data as predictors; (2) uses a sequence deep learning model to fully utilize the sequential nature of historical claims data; (3) can predict the number of duplications of diagnosis related group codes, current procedural terminology codes, and/or international statistical classification of diseases codes in a care path; and (4) uses graph theoretic modeling to correctly order a sequential model output. This is an improvement to prediction systems that generate a sequence of predictions and then orders the predictions to provide an ordered sequence of predictions that may be helpful to a user.

Example inventive and technological advantageous embodiments of the present disclosure include: a composite machine learning model having (1) a machine learning predictive group code model configured to generate a predictive code for an ordered code sequence; (2) a machine learning anchor code model configured to generate an anchor code for an ordered code sequence; (3) a machine learning code sequence model configured to generate an unordered code sequence having a predictive group code, an anchor code, and one or more other codes; and (4) a graphical model configured to generate an ordered code sequence from an unordered code sequence.

### V. Example System Operations

As indicated, various embodiments of the present disclosure make important technical contributions to generating an ordered code sequence. In particular, systems, methods, and computer program products are disclosed herein that implement a composite machine learning model configured to generate an ordered code sequence. In this way, the present disclosure provides a new composite machine learning model and methods for implementing techniques for generating ordered code sequences that improve upon conventional ordered code generation techniques.

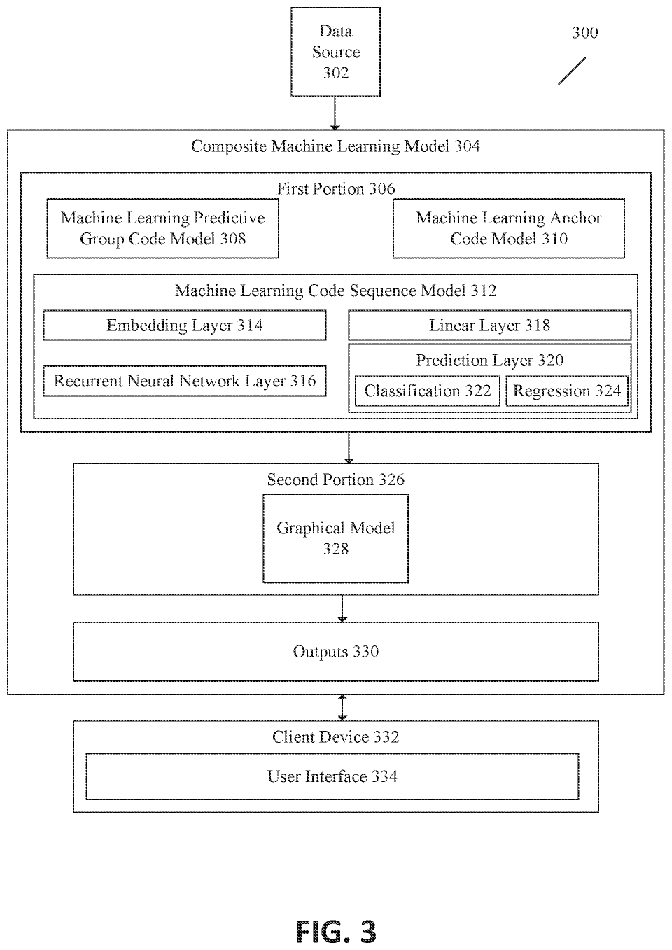

FIG. 3 is an example system diagram 300 showing example systems in accordance with some embodiments discussed herein. The system diagram 300 depicts computing entities for configuring, maintaining, and interacting with a composite machine learning model 304 configured to generate and/or provide one or more ordered code sequences.

The system diagram 300 depicts a composite machine learning model 304 and one or more internal and/or external computing components, such as a client device 332 and a data source 302. The composite machine learning model 304 may include an embodiment of the predictive computing entity 102 and may include one or more components described herein with respect to the predictive computing entity 102. The data source 302 may include one or more computing components of the composite machine learning model 304. Additionally, or alternatively, the data source 302 and/or the client device 332 may be external to the composite machine learning model 304. By way of example, the data source 302 and/or the client device 332 may include embodiments of the external computing entities 112a-c and may include one or more components described herein with respect to the external computing entities 112a-c.

In some embodiments, the client device 332 includes an external computing entity that is configured to interact with the composite machine learning model 304 to generate, maintain, track, and/or the like an ordered code sequence. The client device 332 may be operated by various entities and/or may be associated with, owned by, operated by, and/or the like by one or more end users that may interact with the composite machine learning model 304. For example, a client device 332 may be a personal computing device, smartphone, tablet, laptop, personal digital assistant, and/or the like. In some examples, the one or more end users of a client device 332 may generate, maintain, manage, track, and/or the like an ordered code sequence by leveraging one or functionalities provided by composite machine learning model 304 through user input with the client device 332. By way of example, the client device 332 may include one or more user interfaces 334 (e.g., external I/O elements, etc.) that may be configured to provide one or more application screens presented by one or more computing platforms, such as the composite machine learning model 304. Each of the user interfaces 334, for example, may be configured to present data indicative of an ordered code sequence and/or receive user input indicative of an ordered code sequence, among other inputs.

In some embodiments, the composite machine learning model 304 is configured to generate an ordered code sequence. The composite machine learning model 304 may be configured to generate, maintain, manage, track, and/or the like an ordered code sequence based on entity data.

In some embodiments, the composite machine learning model 304 includes a first portion 306. The first portion 306 of the composite machine learning model 304 may be configured to generate an unordered code sequence based on entity data. The first portion 306 of the composite machine learning model 304 may include one or more of any type of machine learning model including one or more supervised, unsupervised, semi-supervised, and/or reinforcement learning models. The first portion 306 of the composite machine learning model 304 may include multiple models configured to perform one or more different stages of generating an unordered code sequence based on entity data.

In some embodiments, the first portion 306 of the composite machine learning model 304 includes a machine learning predictive group code model 308. The machine learning predictive group code model 308 may be a data entity that describes parameters, hyper-parameters, and/or defined operations of a rules-based and/or machine learning model (e.g., model including at least one of one or more rule-based layers, one or more layers that depend on trained parameters, coefficients, and/or the like). The machine learning predictive group code model 308 may be configured to generate a predictive group code based on entity data. The machine learning predictive group code model 308 may include one or more of any type of machine learning model including one or more supervised, unsupervised, semi-supervised, and/or reinforcement learning models. As an example, the machine learning predictive group code model 308 may include a gradient boosted random forest machine learning model configured to generate a predictive group code based on entity data. The machine learning predictive group code model 308 may include multiple models configured to generate a predictive group code based on entity data (e.g., multiple gradient boosted random forest machine learning models).

In some embodiments, the first portion 306 of the composite machine learning model 304 includes a machine learning anchor code model 310. The machine learning anchor code model 310 may be a data entity that describes parameters, hyper-parameters, and/or defined operations of a rules-based and/or machine learning model (e.g., model including at least one of one or more rule-based layers, one or more layers that depend on trained parameters, coefficients, and/or the like). The machine learning anchor code model 310 may be configured to generate an anchor code based on entity data. The machine learning anchor code model 310 may include one or more of any type of machine learning model including one or more supervised, unsupervised, semi-supervised, and/or reinforcement learning models. As an example, the machine learning anchor code model 310 may include a gradient boosted random forest machine learning model configured to generate an anchor code based on entity data. The machine learning anchor code model 310 may include multiple models configured to generate an anchor code based on entity data (e.g., multiple gradient boosted random forest machine learning models).

In some embodiments, the first portion 306 of the composite machine learning model 304 includes a machine learning code sequence model 312. The machine learning code sequence model 312 may be a data entity that describes parameters, hyper-parameters, and/or defined operations of a rules-based and/or machine learning model (e.g., model including at least one of one or more rule-based layers, one or more layers that depend on trained parameters, coefficients, and/or the like). A machine learning code sequence model 312 may be configured to generate an unordered code sequence based on entity data, an anchor code, and a predictive group code. The machine learning code sequence model 312 may include one or more of any type of machine learning model including one or more supervised, unsupervised, semi-supervised, and/or reinforcement learning models. In some embodiments, the machine learning code sequence model 312 includes multiple models configured to perform one or more different stages of generating an unordered code sequence based on entity data, an anchor code, and a predictive group code.

In some embodiments, the machine learning code sequence model 312 includes an embedding layer 314. The embedding layer 314 may be a data entity that describes parameters, hyper-parameters, and/or defined operations of a rules-based and/or machine learning model (e.g., model including at least one of one or more rule-based layers, one or more layers that depend on trained parameters, coefficients, and/or the like). The embedding layer 314 may be one of a plurality of layers of the machine learning code sequence model 312. The embedding layer 314 may be configured to generate refined entity data based on at least a portion of the entity data. For example, entity data may correspond to a sparse vector Vᵢₙ^{enc}ϵR^{n×1} where n represents a number of entity data values in the entity data and the refined entity data may correspond to a dense vector V_{emb}^{enc}ϵR^{m×1} where n>>m and m represents a portion of the number of entity data values in the refined entity data (e.g., the embedding layer 314 may be configured to map a sparse vector to a dense vector). As an example, in an ordered code sequence prediction domain, n may represent a total number of a patient's prior procedures, prior diagnosis related group codes, prior current procedural terminology codes, prior international statistical classification of diseases codes, demographic features, and/or the like, and/or any other value descriptive of an ordered code sequence process. As an example, in an ordered code sequence prediction domain, m may represent a portion of the number of a patient's prior procedures, prior diagnosis related group codes, prior current procedural terminology codes, prior international statistical classification of diseases codes, demographic features, and/or the like, and/or any other value.

In some embodiments, the machine learning code sequence model 312 includes a recurrent neural network layer 316. The recurrent neural network layer 316 may be a data entity that describes parameters, hyper-parameters, and/or defined operations of a rules-based and/or machine learning model (e.g., model including at least one of one or more rule-based layers, one or more layers that depend on trained parameters, coefficients, and/or the like). The recurrent neural network layer 316 may be one of a plurality of layers of the machine learning code sequence model 312. The recurrent neural network layer 316 may be configured to identify a plurality of predicted codes based on the refined entity data. The recurrent neural network layer 316 may include a plurality of layers. The recurrent neural network layer 316 may generate a hidden state vector V_{hid}^{enc} and an output vector Vₒᵤₜ^{enc} for each of the plurality of layers and the recurrent neural network layer 316 may concatenate the hidden state vector V_{hid}^{enc} from the last layer of the plurality of layers with a R^{2×1} vector to generate a context vector. The context vector may represent the plurality of predicted codes. The R^{2×1} vector may represent at least a portion of the entity data. In some embodiments, the recurrent neural network layer 316 is bidirectional. In some embodiments, the recurrent neural network layer 316 comprises a gated recurrent unit.

In some embodiments, the machine learning code sequence model 312 includes a linear layer 318. The linear layer 318 may be a data entity that describes parameters, hyper-parameters, and/or defined operations of a rules-based and/or machine learning model (e.g., model including at least one of one or more rule-based layers, one or more layers that depend on trained parameters, coefficients, and/or the like). The linear layer 318 may be configured to identify a number of predicted codes in an unordered code sequence based on a plurality of predicted codes and at least a portion of the entity data. As one example, in an ordered code sequence prediction domain, the linear layer 318 may identify a number of diagnosis related group codes, current procedural terminology codes, and/or international statistical classification of diseases codes in an unordered code sequence.

In some embodiments, the machine learning code sequence model 312 includes a prediction layer 320. The prediction layer 320 may be a data entity that describes parameters, hyper-parameters, and/or defined operations of a rules-based and/or machine learning model (e.g., model including at least one of one or more rule-based layers, one or more layers that depend on trained parameters, coefficients, and/or the like).

In some embodiments, the prediction layer 320 includes a classification model 322. The classification model 322 may be a data entity that describes parameters, hyper-parameters, and/or defined operations of a rules-based and/or machine learning model (e.g., model including at least one of one or more rule-based layers, one or more layers that depend on trained parameters, coefficients, and/or the like). The classification model 322 may be configured to identify one or more predicted codes in an unordered code sequence from a plurality of predicted codes. For example, the classification model 322 may be configured to identify one or more predicted codes in an unordered code sequence from a plurality of predicted codes using a sigmoid activation function.

The classification model 322 (e.g., using a sigmoid activation function) may be configured to identify the one or more predicted codes in an unordered code sequence from a plurality of codes by determining which of the plurality of codes is associated with a probability of being in an unordered code sequence that exceeds a probability threshold. For example, the classification model 322 (e.g., using a sigmoid activation function) may be configured to identify the one or more predicted codes in an unordered code sequence from a plurality of codes by determining which of the plurality of codes is associated with a probability of being in an unordered code sequence that exceeds a probability threshold of 0.5. In some embodiments, the classification model 322 is trained using a binary cross-entropy technique. Additionally, or alternatively, the classification model 322 is trained using a root-mean-squared error technique. As one example, in an ordered code sequence prediction domain, the classification model 322 may identify one or more diagnosis related group codes, current procedural terminology codes, and/or international statistical classification of diseases codes in an unordered code sequence.

In some embodiments, the prediction layer 320 may include a regression model 324. The regression model 324 may be a data entity that describes parameters, hyper-parameters, and/or defined operations of a rules-based and/or machine learning model (e.g., model including at least one of one or more rule-based layers, one or more layers that depend on trained parameters, coefficients, and/or the like). The regression model 324 may be configured to identify an occurrence of each of one or more predicted codes in an unordered code sequence. For example, the regression model 324 may be configured to identify an occurrence of each of the one or more predicted codes in an unordered code sequence using a rectified linear unit activation function.

The regression model 324 (e.g., using a rectified linear unit activation function) may be configured to determine an occurrence of each of one or more predicted codes in an unordered code sequence by determining a count associated with each code in an unordered code sequence. For example, the regression model 324 (e.g., using a rectified linear unit activation function) may be configured to determine an occurrence of a predicted code in an unordered code sequence by determining that the predicted code is associated with a count of 3 (e.g., the predicted code occurs 3 times in the unordered code sequence). In some embodiments, the regression model 324 is trained using a binary cross-entropy technique. Additionally, or alternatively, the regression model 324 is trained using a root-mean-squared error technique. As one example, in an ordered code sequence prediction domain, the regression model 324 may identify an occurrence of each of one or more diagnosis related group codes, current procedural terminology codes, and/or international statistical classification of diseases codes in an unordered code sequence.

In some embodiments, the composite machine learning model 304 includes a second portion 326. The second portion 326 of the composite machine learning model 304 may be configured to generate an ordered code sequence based on an ordered code sequence. The second portion 326 of the composite machine learning model 304 may include one or more of any type of machine learning model including one or more supervised, unsupervised, semi-supervised, and/or reinforcement learning models. The second portion 326 of the composite machine learning model 304 may include multiple models configured to perform one or more different stages of generating an ordered code sequence based on an unordered code sequence.

In some embodiments, the second portion 326 of the composite machine learning model 304 includes a graphical model 328. The graphical model 328 may be a data entity that describes parameters, hyper-parameters, and/or defined operations of a rules-based and/or machine learning model (e.g., model including at least one of one or more rule-based layers, one or more layers that depend on trained parameters, coefficients, and/or the like). The graphical model 328 may be configured to generate an ordered code sequence based on an unordered code sequence. The graphical model 328 may include one or more of any type of machine learning model including one or more supervised, unsupervised, semi-supervised, and/or reinforcement learning models. As an example, the graphical model 328 may include a transition probably matrix configured to generate the ordered code sequence. The graphical model 328 may include multiple models configured to generate the ordered code sequence based on the unordered code sequence.

In some embodiments, the composite machine learning model 304 is configured to provide one or more outputs 330. For example, the composite machine learning model 304 may be configured to provide an ordered code sequence as the one or more outputs 330. In some embodiments, providing the one or more outputs 330 includes causing the one or more outputs 330 to be displayed on the user interface 335 of the client device 332. In some embodiments, providing the one or more outputs 330 includes initiating performance of one or more prediction-based actions

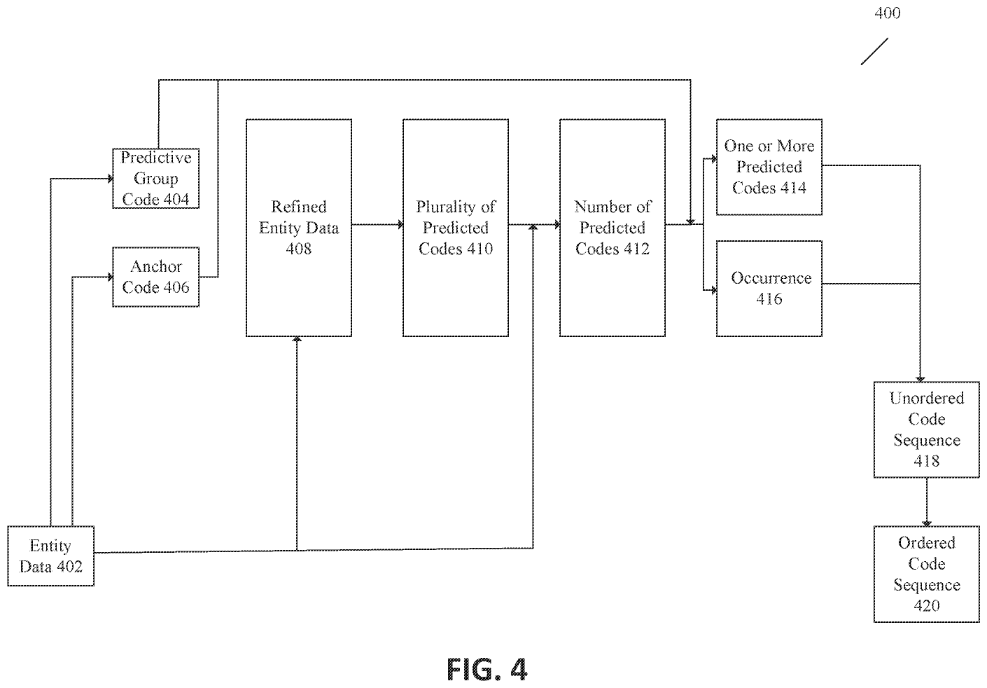

FIG. 4 is a dataflow diagram 400 showing example data structures for facilitating the generation of an ordered code sequence in accordance with some embodiments discussed herein. The dataflow diagram 400 depicts a set of data structures and algorithms for facilitating generation of an ordered code sequence.

In some embodiments, the entity data 402 is a data entity that describes data provided to an algorithm to generate a predictive output. Entity data 402 may include a plurality of entity data values that may be considered by an algorithm to generate the predictive output. In some embodiments, the algorithm is a machine learning model that may be trained to generate the predictive output based on the entity data values of the entity data 402. As one example, in an ordered code sequence prediction domain, entity data 402 may correspond to one or more medical records associated with a patient. In such a case, entity data 402 may include one or more entity data values that describe a patient's prior procedures, prior diagnosis related group codes, prior current procedural terminology codes, prior international statistical classification of diseases codes, demographic features, and/or the like, and/or any other value descriptive of an ordered code sequence process.

In some embodiments, an entity may be a data entity that describes an entity corresponding to entity data 402. An entity may include a plurality of entity values that may be considered by an algorithm to generate a predictive output. In some examples, the algorithm may include a machine learning model that is trained to generate a predictive output for entity data 402 based on one or more entity values of a corresponding entity. As one example, in an ordered code sequence prediction domain, an entity may correspond to a patient for which an ordered code sequence may be generated.

In some embodiments, the machine learning predictive group code model 308 is configured to generate a predictive group code 404 based on the entity data 402. As one example, in an ordered code sequence prediction domain, a predictive group code 404 may correspond to a diagnosis related group code corresponding to an entity for which an ordered code sequence may be generated. For example, a predictive group code 404 may correspond to an outpatient procedure associated with an entity for which an ordered code sequence may be generated. In some embodiments, the predictive group code 404 is one of the codes in the unordered code sequence 418 and/or the ordered code sequence 420.

In some embodiments, the machine learning anchor code model 310 is configured to generate an anchor code 406 based on the entity data 402. In some embodiments, anchor code 406 is one of the codes in the unordered code sequence 418 and/or the ordered code sequence 420. In some embodiments, the anchor code 406 is the first code in the unordered code sequence 418 and/or the ordered code sequence 420. As one example, in an ordered code sequence prediction domain, an anchor code 406 may correspond to a current procedural terminology code and/or an international statistical classification of diseases code corresponding to an entity for which an ordered code sequence may be generated.

In some embodiments, the embedding layer 314 of the machine learning code sequence model 312 is configured to generate refined entity data 408 based on at least a portion of the entity data 402. The refined entity data 408 may be a data entity that describes data provided to an algorithm to generate a predictive output. Refined entity data 408 may include a plurality of refined entity data values that may be considered by an algorithm to generate the predictive output. In some embodiments, the algorithm is a machine learning model that may be trained to generate the predictive output based on the refined entity data values of the refined entity data 408. As one example, in an ordered code sequence prediction domain, refined entity data 408 may correspond to a portion of one or more medical records associated with a patient. In such a case, refined entity data 408 may include one or more refined entity data values that describe a portion of a patient's prior procedures, prior diagnosis related group codes, prior current procedural terminology codes, prior international statistical classification of diseases codes, demographic features, and/or the like, and/or any other value descriptive of an ordered code sequence process.

In some embodiments, the recurrent neural network layer 316 of the machine learning code sequence model 312 is configured to identify a plurality of predicted codes 410 based on the refined entity data 408. The plurality of predicted codes 410 may be a collection of one or more codes. As one example, in an ordered code sequence prediction domain, a plurality of predicted codes 410 may include diagnosis related group codes, current procedural terminology codes, and/or international statistical classification of diseases codes.

In some embodiments, the linear layer 318 of the machine learning code sequence model 312 is configured to determine a number of predicted codes 412 in the one or more predicted codes 414 based on the plurality of predicted codes 410 and/or at least a portion of the entity data 402. For example, the linear layer 318 may be configured to determine that the number of predicted codes 412 in the one or more predicted codes 414 is less than the number of predicted codes in the plurality of predicted codes 410.

In some embodiments, the classification model 322 of the prediction layer 320 of the machine learning code sequence model 312 is configured to identify one or more predicted codes 414 in the unordered code sequence 418. For example, the classification model 322 may be configured to identify the one or more predicted codes 414 in the unordered code sequence 418 from a plurality of predicted codes 410 using a sigmoid activation function. The one or more predicted codes 414 may be codes that are in the unordered code sequence 418 and/or the ordered code sequence 420. In some embodiments, the one or more predicted codes 414 are distinct from the plurality of predicted codes 410 in that all of the one or more predicted codes 414 are in the unordered code sequence 418 and/or an ordered code sequence 420 while not all of the plurality of predicted codes 410 are in the unordered code sequence 418 and/or an ordered code sequence 420. As one example, in an ordered code sequence prediction domain, the classification model 322 may identify one or more diagnosis related group codes, current procedural terminology codes, and/or international statistical classification of diseases codes in an unordered code sequence 418 and/or ordered code sequence 420.

In some embodiments, the regression model 324 of the prediction layer 320 of the machine learning code sequence model 312 is configured to determine an occurrence 416 of each of the one or more predicted codes 414 in the unordered code sequence 418 and/or the ordered code sequence 420. For example, the regression model 324 may be configured to identify an occurrence 416 of each of the one or more predicted codes 414 in the unordered code sequence 418 using a rectified linear unit activation function. For example, the regression model 324 may determine the number of times each of the one or more predicted codes 414 occur in the unordered code sequence 418 and/or the ordered code sequence 420.

In some embodiments, the unordered code sequence 418 is a sequence of codes generated by the machine learning code sequence model 312 that are not organized in a particular determined order. As one example, in an ordered code sequence prediction domain, the unordered code sequence 418 may include a sequence of diagnosis related group codes, current procedural terminology codes, and/or international statistical classification of diseases codes that are not organized in a particular determined order.

In some embodiments, the graphical model 328 of the composite machine learning model 304 may be configured to determine an ordered code sequence 420 based on the unordered code sequence 418. For example, the graphical model 328 may include a transition probably matrix configured to generate the ordered code sequence 420. The ordered code sequence 420 may be a sequence of codes that are organized in a particular determined order. As one example, in an ordered code sequence prediction domain, the ordered code sequence 420 may include a sequence of diagnosis related group codes, current procedural terminology codes, and/or international statistical classification of diseases codes that are organized in a particular determined order.

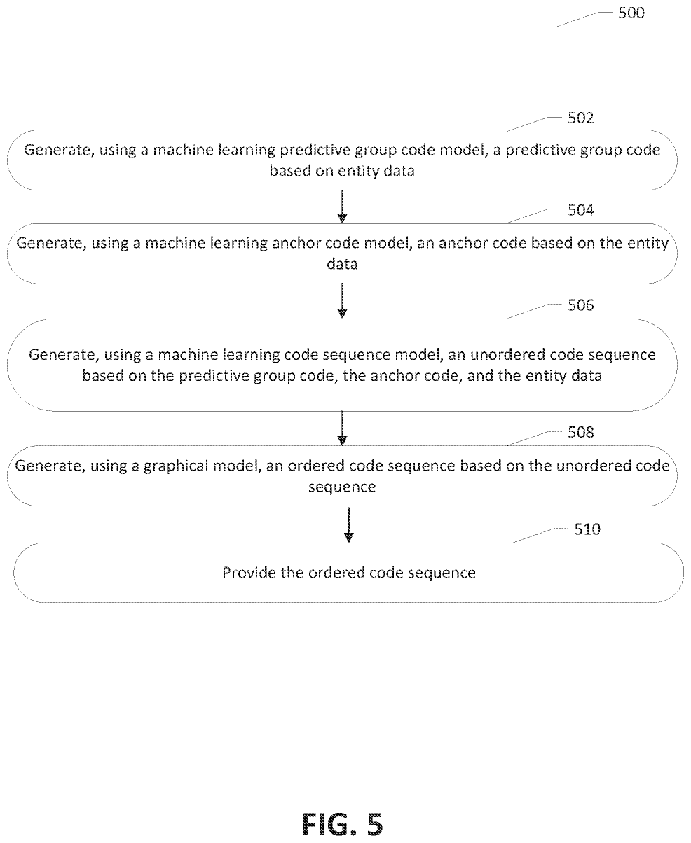

FIG. 5 is a flowchart showing an example of a process 500 for generating an ordered code sequence based on entity data in accordance with some embodiments discussed herein. The flowchart depicts techniques for generating an ordered code sequence based on entity data. The techniques may be implemented by one or more computing devices, entities, and/or systems described herein. For example, via the various steps/operations of the process 500, the computing system 100 may leverage a composite machine learning model to overcome the various limitations with conventional techniques for generating an ordered code sequence that are (i) inefficient (e.g., resource intensive and/or time consuming), (ii) lack scalability for large ordered sequences and/or large quantities of entity data, and/or (iii) generate inaccurate ordered code sequences.

FIG. 5 illustrates an example process 500 for explanatory purposes. Although the example process 500 depicts a particular sequence of steps/operations, the sequence may be altered without departing from the scope of the present disclosure. For example, some of the steps/operations depicted may be performed in parallel or in a different sequence that does not materially impact the function of the process 500. In other examples, different components of an example device or system that implements the process 500 may perform functions at substantially the same time or in a specific sequence.

In some embodiments, the process 500 includes, at step/operation 502, generating, using a machine learning predictive group code model, a predictive group code based on entity data. For example, the computing system 100, using the machine learning predictive group code model, may generate the predictive group code based on the entity data. The entity data may be a data entity that describes data provided to an algorithm to generate a predictive output. For example, the entity data may include one or more entity data values that describe a patient's prior procedures, prior diagnosis related group codes, prior current procedural terminology codes, prior international statistical classification of diseases codes, demographic features, and/or the like, and/or any other value descriptive of an ordered code sequence process.

In some embodiments, the predictive group code is one of the codes in the unordered code sequence and/or the ordered code sequence. For example, the predictive group code may correspond to a diagnosis related group code corresponding to an entity for which an ordered code sequence may be generated. For example, the predictive group code may correspond to an outpatient procedure associated with an entity for which an ordered code sequence may be generated.

In some embodiments, the process 500 includes, at step/operation 504, generating, using a machine learning anchor code model, an anchor based on the entity data. For example, the computing system 100, using the machine learning anchor code model, may generate the anchor code based on the entity data. In some embodiments, anchor code is one of the codes in the unordered code sequence and/or the ordered code sequence. In some embodiments, the anchor code is the first code in the unordered code sequence and/or the ordered code sequence. For example, the anchor code may correspond to a current procedural terminology code and/or an international statistical classification of diseases code corresponding to an entity for which an ordered code sequence may be generated.

In some embodiments, the process 500 includes, at step/operation 506, generating, using a machine learning code sequence model, an unordered code sequence based on the predictive group code, the anchor code, and the entity data. For example, the computing system 100, using the machine learning code sequence model, may generate the unordered code sequence based on the predictive group code, the anchor code, and the entity data.

In some embodiments, the machine learning code sequence model includes an embedding layer. In some embodiments, the embedding layer of the machine learning code sequence model is configured to generate refined entity data based on at least a portion of the entity data. The refined entity data may be a data entity that describes data provided to an algorithm to generate a predictive output. Refined entity data may include a plurality of refined entity data values that may be considered by an algorithm to generate the predictive output. In some embodiments, the algorithm is a machine learning model that may be trained to generate the predictive output based on the refined entity data values of the refined entity data. As one example, in an ordered code sequence prediction domain, refined entity data may correspond to a portion of one or more medical records associated with a patient. In such a case, refined entity data may include one or more refined entity data values that describe a portion patient's prior procedures, prior diagnosis related group codes, prior current procedural terminology codes, prior international statistical classification of diseases codes, demographic features, and/or the like, and/or any other value descriptive of an ordered code sequence process.

In some embodiments, the machine learning code sequence model may include a recurrent neural network layer. In some embodiments, the recurrent neural network layer of the machine learning code sequence model is configured to identify a plurality of predicted codes based on the refined entity data. The plurality of predicted codes may be a collection of one or more codes. As one example, in an ordered code sequence prediction domain, a plurality of predicted codes may include diagnosis related group codes, current procedural terminology codes, and/or international statistical classification of diseases codes.

In some embodiments, the machine learning code sequence model may include a linear layer. In some embodiments, the linear layer of the machine learning code sequence model is configured to determine a number of predicted codes in the one or more predicted codes based on the plurality of predicted codes and/or at least a portion of the entity data. For example, the linear layer may be configured to determine that the number of predicted codes in the one or more predicted codes is less than the number of predicted codes in the plurality of predicted codes.

In some embodiments, the machine learning code sequence model may include a prediction layer having a classification model and a regression model. In some embodiments, the classification model of the prediction layer of the machine learning code sequence model is configured to identify one or more predicted codes in the unordered code sequence. For example, the classification model may be configured to identify the one or more predicted codes in the unordered code sequence from a plurality of predicted codes using a sigmoid activation function. The one or more predicted codes may be codes that are in the unordered code sequence and/or the ordered code sequence. In some embodiments, the one or more predicted codes are distinct from the plurality of predicted codes in that all of the one or more predicted codes are in the unordered code sequence and/or an ordered code sequence while not all of the plurality of predicted codes are in the unordered code sequence and/or an ordered code sequence. As one example, in an ordered code sequence prediction domain, the classification model may identify one or more diagnosis related group codes, current procedural terminology codes, and/or international statistical classification of diseases codes in an unordered code sequence and/or ordered code sequence.

In some embodiments, the regression model of the prediction layer of the machine learning code sequence model is configured to determine an occurrence of each of the one or more predicted codes in the unordered code sequence and/or the ordered code sequence. For example, the regression model may be configured to identify an occurrence of each of the one or more predicted codes in the unordered code sequence using a rectified linear unit activation function. For example, the regression model may determine the number of times each of the one or more predicted codes occur in the unordered code sequence and/or the ordered code sequence.

In some embodiments, the unordered code sequence is a sequence of codes generated by the machine learning code sequence model that are not organized in a particular determined order. As one example, in an ordered code sequence prediction domain, the unordered code sequence may include a sequence of diagnosis related group codes, current procedural terminology codes, and/or international statistical classification of diseases codes that are not organized in a particular determined order.

In some embodiments, the process 500 includes, at step/operation 508, generating, using a graphical model, an ordered code sequence based on the unordered code sequence. For example, the computing system 100, using the graphical model, may generate the ordered code sequence based on the unordered code sequence.

In some embodiments, the graphical model of the composite machine learning model may be configured to determine an ordered code sequence based on the unordered code sequence. For example, the graphical model may include a transition probably matrix configured to generate the ordered code sequence. The ordered code sequence may be a sequence of codes that are organized in a particular determined order. As one example, in an ordered code sequence prediction domain, the ordered code sequence may include a sequence of diagnosis related group codes, current procedural terminology codes, and/or international statistical classification of diseases codes that are organized in a particular determined order.

The composite machine learning model presents machine learning and data generation techniques that improve computer generation of ordered code sequences. To do so, the composite machine learning model leverages multiple types of machine learning models and/or rules-based models to generate an ordered code sequence based on input data in an efficient, accurate, and scalable manner. In this way, the present disclosure provides a new composite machine learning model and methods for implementing techniques for generating ordered code sequences that improve upon conventional ordered code generation techniques.

In some embodiments, the process 500 includes, at step/operation 510, providing the ordered code sequence. For example, the computing system 100 may provide the ordered code sequence. In some embodiments, providing the ordered code sequence includes causing the ordered code sequence to be displayed on a user interface. In some embodiments, providing the ordered code sequences includes initiating performance of one or more prediction-based actions.

Some techniques of the present disclosure enable the generation of action outputs that may be performed to initiate one or more prediction-based actions to achieve real-world effects. The multi-phase training techniques of the present disclosure may be used, applied, and/or otherwise leveraged to generate a composite machine learning model, which may help in the computer generation of ordered code sequences. The composite machine learning model of the present disclosure may be leveraged to initiate the performance of various computing tasks that improve the performance of a computing system (e.g., a computer itself, etc.) with respect to various prediction-based actions performed by the computing system 100, such as for the generation of ordered code sequences and/or the like. Example prediction-based actions may include the generation of an ordered code sequence.

In some examples, the computing tasks may include prediction-based actions that may be based on a prediction domain. A prediction domain may include any environment in which computing systems may be applied to achieve real-word insights, such as predictions (e.g., ordered code sequences, etc.), and initiate the performance of computing tasks, such as prediction-based actions e.g., updating user preferences, providing account information, cancelling an account, adding an account, etc.) to act on the real-world insights. These prediction-based actions may cause real-world changes, for example, by controlling a hardware component, providing alerts, interactive actions, and/or the like.

Examples of prediction domains may include, clinical systems, autonomous systems, and/or the like. Prediction-based actions in such domains may include the initiation of automated instructions across and between devices, automated notifications, automated scheduling operations, automated precautionary actions, automated security actions, automated data processing actions, automated data compliance actions, automated data access enforcement actions, automated adjustments to computing and/or human data access management, and/or the like.

In some embodiments, the multi-phase techniques of process 500 are applied to initiate the performance of one or more prediction-based actions. A prediction-based action may depend on the prediction domain. In some examples, the computing system 100 may leverage the multi-stage techniques to generate a composite machine learning model that may be leveraged to initiate the generation of an ordered code sequence, and/or any other related operations.

### VI. Conclusion

Many modifications and other embodiments will come to mind to one skilled in the art to which the present disclosure pertains having the benefit of the teachings presented in the foregoing descriptions and the associated drawings. Therefore, it is to be understood that the present disclosure is not to be limited to the specific embodiments disclosed and that modifications and other embodiments are intended to be included within the scope of the appended claims. Although specific terms are employed herein, they are used in a generic and descriptive sense only and not for purposes of limitation.

### VII. Examples

Example 1. A computer-implemented method comprising: generating, by one or more processors and using a first portion of a composite machine learning model, a predictive group code and an anchor code for an entity based on entity data; generating, by the one or more processors and using the first portion of the composite machine learning model, an unordered code sequence comprising one or more predicted codes based on the entity data, the predictive group code, and the anchor code; generating, by the one or more processors and using a second portion of the composite machine learning model, an ordered code sequence based on the unordered code sequence; and providing, by the one or more processors, the ordered code sequence.

Example 2. The computer-implemented method of example 1, wherein the first portion of the composite machine learning model comprises a plurality of machine learning models, wherein each of the plurality of machine learning models is configured to generate one of the predictive group code, the anchor code, and the unordered code sequence.

Example 3. The computer-implemented method of example 2, wherein the plurality of machine learning models comprises a machine learning code sequence model configured to generate the unordered code sequence, wherein the machine learning code sequence model comprises an embedding layer configured to generate refined entity data based on at least a portion of the entity data.

Example 4. The computer-implemented method of example 3, wherein the machine learning code sequence model further comprises a recurrent neural network layer configured to identify a plurality of predicted codes wherein the recurrent neural network layer comprises a gated recurrent unit, wherein the recurrent neural network layer is bidirectional.

Example 5. The computer-implemented method of example 4, wherein the machine learning code sequence model further comprises a linear layer configured to determine a number of predicted codes in the one or more predicted codes based on the plurality of predicted codes and at least a portion of the entity data.

Example 6. The computer-implemented method of any of examples 4 through 5, wherein the machine learning code sequence model further comprises a prediction layer.

Example 7. The computer-implemented method of example 4, wherein the prediction layer comprises a classification model configured to identify the one or more predicted codes in the unordered code sequence from the plurality of predicted codes.

Example 8. The computer-implemented method of example 4, wherein the prediction layer comprises a regression model configured to determine an occurrence of each of the one or more predicted codes in the unordered code sequence.

Example 9. The computer-implemented method of any of examples 2 through 8, wherein the plurality of machine learning models comprises a machine learning predictive group code model configured to generate the predictive group code.

Example 10. The computer-implemented method of any of examples 2 through 9, wherein the plurality of machine learning models comprises a machine learning anchor code model configured to generate the anchor code.

Example 11. The computer-implemented method of any of the preceding examples, wherein the second portion of the composite machine learning model comprises a graphical model configured to generate the ordered code sequence, wherein the graphical model comprises a transition probability matrix.

Example 12. A computing system comprising memory and one or more processors communicatively coupled to the memory, the one or more processors configured to: generate, using a first portion of a composite machine learning model, a predictive group code and an anchor code for an entity based on entity data; generate, using the first portion of the composite machine learning model, an unordered code sequence comprising one or more predicted codes based on the entity data, the predictive group code, and the anchor code; generate, using a second portion of the composite machine learning model, an ordered code sequence based on the unordered code sequence; and provide the ordered code sequence

Example 13. The computing system of example 12, wherein the first portion of the composite machine learning model comprises a plurality of machine learning models, wherein each of the plurality of machine learning models is configured to generate one of the predictive group code, the anchor code, and the unordered code sequence

Example 14. The computing system of example 13, wherein the plurality of machine learning models comprises a machine learning code sequence model configured to generate the unordered code sequence, wherein the machine learning code sequence model comprises an embedding layer configured to generate refined entity data based on at least a portion of the entity data

Example 15. The computing system of example 14, wherein the machine learning code sequence model further comprises a recurrent neural network layer configured to identify a plurality of predicted codes wherein the recurrent neural network layer comprises a gated recurrent unit, wherein the recurrent neural network layer is bidirectional, wherein the machine learning code sequence model further comprises a linear layer configured to determine a number of predicted codes in the one or more predicted codes based on the plurality of predicted codes and at least a portion of the entity data

Example 16. The computing system of example 15, wherein the machine learning code sequence model further comprises a prediction layer, wherein the prediction layer comprises a classification model configured to identify the one or more predicted codes in the unordered code sequence from the plurality of predicted codes, wherein the prediction layer comprises a regression model configured to determine an occurrence of each of the one or more predicted codes in the unordered code sequence.

Example 17. The computing system of any of examples 13 through 16, wherein the plurality of machine learning models comprises a machine learning predictive group code model configured to generate the predictive group code.

Example 18. The computing system of any of examples 13 through 17, wherein the plurality of machine learning models comprises a machine learning anchor code model configured to generate the anchor code.

Example 19. The computing system of any of any of the preceding examples, wherein the second portion of the composite machine learning model comprises a graphical model configured to generate the ordered code sequence, wherein the graphical model comprises a transition probability matrix.

Example 20. One or more non-transitory computer-readable storage media including instructions that, when executed by one or more processors, cause the one or more processors to: generate, using a first portion of a composite machine learning model, a predictive group code and an anchor code for an entity based on entity data; generate, using the first portion of the composite machine learning model, an unordered code sequence comprising one or more predicted codes based on the entity data, the predictive group code, and the anchor code; generate, using a second portion of the composite machine learning model, an ordered code sequence based on the unordered code sequence; and provide the ordered code sequence.

1. A computer-implemented method comprising:
generating, by one or more processors and using a first portion of a composite machine learning model, a predictive group code and an anchor code for an entity based on entity data;
generating, by the one or more processors and using the first portion of the composite machine learning model, an unordered code sequence comprising one or more predicted codes based on the entity data, the predictive group code, and the anchor code;
generating, by the one or more processors and using a second portion of the composite machine learning model, an ordered code sequence based on the unordered code sequence; and
providing, by the one or more processors, the ordered code sequence.

2. The computer-implemented method of claim 1, wherein the first portion of the composite machine learning model comprises a plurality of machine learning models, wherein each of the plurality of machine learning models is configured to generate one of the predictive group code, the anchor code, and the unordered code sequence.

3. The computer-implemented method of claim 2, wherein the plurality of machine learning models comprises a machine learning code sequence model configured to generate the unordered code sequence, wherein the machine learning code sequence model comprises an embedding layer configured to generate refined entity data based on at least a portion of the entity data.

4. The computer-implemented method of claim 3, wherein the machine learning code sequence model further comprises a recurrent neural network layer configured to identify a plurality of predicted codes wherein the recurrent neural network layer comprises a gated recurrent unit, wherein the recurrent neural network layer is bidirectional.

5. The computer-implemented method of claim 4, wherein the machine learning code sequence model further comprises a linear layer configured to determine a number of predicted codes in the one or more predicted codes based on the plurality of predicted codes and at least a portion of the entity data.

6. The computer-implemented method of claim 4, wherein the machine learning code sequence model further comprises a prediction layer.

7. The computer-implemented method of claim 6, wherein the prediction layer comprises a classification model configured to identify the one or more predicted codes in the unordered code sequence from the plurality of predicted codes.

8. The computer-implemented method of claim 6, wherein the prediction layer comprises a regression model configured to determine an occurrence of each of the one or more predicted codes in the unordered code sequence.

9. The computer-implemented method of claim 2, wherein the plurality of machine learning models comprises a machine learning predictive group code model configured to generate the predictive group code.

10. The computer-implemented method of claim 2, wherein the plurality of machine learning models comprises a machine learning anchor code model configured to generate the anchor code.

11. The computer-implemented method of claim 1, wherein the second portion of the composite machine learning model comprises a graphical model configured to generate the ordered code sequence, wherein the graphical model comprises a transition probability matrix.

12. A computing system comprising memory and one or more processors communicatively coupled to the memory, the one or more processors configured to:
generate, using a first portion of a composite machine learning model, a predictive group code and an anchor code for an entity based on entity data;
generate, using the first portion of the composite machine learning model, an unordered code sequence comprising one or more predicted codes based on the entity data, the predictive group code, and the anchor code;
generate, using a second portion of the composite machine learning model, an ordered code sequence based on the unordered code sequence; and
provide the ordered code sequence.

13. The computing system of claim 12, wherein the first portion of the composite machine learning model comprises a plurality of machine learning models, wherein each of the plurality of machine learning models is configured to generate one of the predictive group code, the anchor code, and the unordered code sequence.

14. The computing system of claim 13, wherein the plurality of machine learning models comprises a machine learning code sequence model configured to generate the unordered code sequence, wherein the machine learning code sequence model comprises an embedding layer configured to generate refined entity data based on at least a portion of the entity data.

15. The computing system of claim 14, wherein the machine learning code sequence model further comprises a recurrent neural network layer configured to identify a plurality of predicted codes wherein the recurrent neural network layer comprises a gated recurrent unit, wherein the recurrent neural network layer is bidirectional, wherein the machine learning code sequence model further comprises a linear layer configured to determine a number of predicted codes in the one or more predicted codes based on the plurality of predicted codes and at least a portion of the entity data.

16. The computing system of claim 15, wherein the machine learning code sequence model further comprises a prediction layer, wherein the prediction layer comprises a classification model configured to identify the one or more predicted codes in the unordered code sequence from the plurality of predicted codes, wherein the prediction layer comprises a regression model configured to determine an occurrence of each of the one or more predicted codes in the unordered code sequence.

17. The computing system of claim 13, wherein the plurality of machine learning models comprises a machine learning predictive group code model configured to generate the predictive group code.

18. The computing system of claim 13, wherein the plurality of machine learning models comprises a machine learning anchor code model configured to generate the anchor code.

19. The computing system of claim 12, wherein the second portion of the composite machine learning model comprises a graphical model configured to generate the ordered code sequence, wherein the graphical model comprises a transition probability matrix.

20. One or more non-transitory computer-readable storage media including instructions that, when executed by one or more processors, cause the one or more processors to:
generate, using a first portion of a composite machine learning model, a predictive group code and an anchor code for an entity based on entity data;
generate, using the first portion of the composite machine learning model, an unordered code sequence comprising one or more predicted codes based on the entity data, the predictive group code, and the anchor code;
generate, using a second portion of the composite machine learning model, an ordered code sequence based on the unordered code sequence; and
provide the ordered code sequence.
